(19) United States
(12) Patent Application Publication
(10) Pub. No.: US 2004/0254551 A1
(43) Pub. Date: Dec. 16, 2004

(54) [ABSORBENT CORE STRUCTURE FOR HYGIENE PRODUCTS ]

(75) Inventors: Keith J. Carnes, Concord, NC (US); David A. Murillo, Charlotte, NC (US)

Correspondence Address: SUMMA & ALLAN, P.A., 11610 NORTH COMMUNITY HOUSE ROAD, SUITE 200, CHARLOTTE, NC, 28277, US

(73) Assignee: WELLMAN, INC, Charlotte, NC (US)

(21) Appl. No.: 10/250,191

(22) Filed: Jun. 11, 2003

(51) Int. Cl. A61F 13/15; A61F 13/20

(52) U.S. Cl. 604/367

(57) ABSTRACT

An absorbent core structure is disclosed for acquiring and absorbing aqueous-based liquids in hygiene products. The core structure includes a nonwoven blend of fluff pulp for wicking and absorbing an aqueous-based liquid, a superabsorbent polymer for absorbing an aqueous-based liquid and retaining the liquid against moderate pressure against the core structure, a binding element or structure (such as a synthetic bicomponent filament) for providing at least some of the bonding in the nonwoven blend, and a plurality of small denier hollow filaments for enhancing the moisture transport and storage of the liquid and improving the physical structure as compared to fluff.

### BACKGROUND OF INVENTION

The present invention relates to fluid-absorbing hygiene products, and in particular relates to core structures for such products which typically include baby diapers, adult incontinence garments, and feminine hygiene pads. Each of these products typically includes an absorbent core for absorbing and containing body fluids, with the cores being structurally combined with other elements that relate to the fit, odor control, comfort and similar functions of the absorbent item.

In almost every case, the diaper (or other absorbent structure) should desirably perform several related functions. First, the structure should take up the desired fluid, a step typically referred to as “acquisition” or “uptake.” Second, the structure should have the capacity to hold the liquid taken up, a property referred to as “absorbency.” Third, the structure should have the capacity to maintain the liquid for a desired period of time and against moderate pressure (e.g., a baby with a wet diaper sitting in a car seat), a property referred to as “retention”.

The conventional cores for such products are currently typically formed of a nonwoven composite of fluff pulp (cellulose fibers) and super absorbent polymer (SAP). In most circumstances, synthetic fibers such as polyester are absent from the core, with the exception that some bicomponent synthetic fibers are often included for bonding purposes.

The term “nonwoven” is used in its conventional sense to describe sheet or web structures bonded together by entangling fiber or filaments mechanically, thermally, or chemically. Nonwovens are typically flat porous sheets made directly from separate fibers. As their name implies, they are not made by weaving or knitting and do not require converting the fibers to yarn.

As used herein, a super absorbent polymer (“SAP”) is a polymer that absorbs at least about 40 times its weight in water or similar aqueous-based fluids. Super absorbent polymers are typically cross-linked polyelectrolytes and their chemistry, structures, and functions are generally well understood in this art. Super absorbent polymers and other powders enhance absorbency, control odor and help ensure effective fluid retention.

Fluff (or “fluff pulp) is the term used to describe cellulose fibers (e.g. natural fibers from cotton and wood) in this art and these are typically incorporated to provide absorbency and some wicking. Such natural wood and cotton fibers also provide consumer appeal in products such as paper towels and facial and bathroom tissue.

The total hygiene market (for core materials) is currently about 9.3 billion pounds per year with retail sales representing a dollar multiple of that figure, currently about $36 billion dollars. Of this market, about 8.9 billion pounds are run on conventional forming machines, with about 400 million pounds being formed on airlaid core machines. This usage can also be expressed as about 7.7 billion pounds of fluff and about 1.6 billion pounds of SAP.

Of these main groups of hygiene products, baby diapers comprise the largest and most competitive portion. The technology is either closely held confidential material, or covered by a large number of patents in a crowded field. At present, however, very little of this activity relates to the proprietary nature of the basic core, which is generally well understood. Instead, the proprietary nature of diaper design and manufacturing relates more to the machines used to produce them.

In particular, the cores of most commercially available baby diapers are formed in place; i.e., the individual core for a single diaper is formed along with the diaper itself (“in situ”). This process, although advantageous, presents certain disadvantages. In particular, diapers have cores that are about three inches wide and ten or twelve inches long. Accordingly, because the cores are formed concurrently with the diapers, the machinery that makes the diapers must be capable of depositing a small amount of core material in this size onto each In turn, making diapers at a relatively high rate requires a relatively large number of machines. The current commercial rate of diaper production is about 300 per minute.

Furthermore, the nature of fluff's acquisition properties are such that a fluff-based core typically requires one or more separate acquisition and distribution layers. In turn, in commercial manufacturing, adding each additional layer or structural element (elastic trim, fastening tabs, etc.) requires an additional source or supply, and an additional machine or machine portion to add the layer or other element as the diaper is being formed. In short, modern diaper construction and manufacturing are very complex.

Accordingly, interest has been generated in other core materials that can simplify and thus streamline and increase the speed and efficiency of the manufacturing process. One such type of nonwoven material is referred to as “airlaid.” An airlaid material and process offers a number of advantages, particularly to the diaper manufacturer, even if the end product that reaches the consumer may not strike the consumer as being any different.

The term airlaid is used in this art to both name a particular process for making nonwoven webs and to describe the resulting nonwoven structures. The term will be used in both senses herein, with the meaning in any particular case being clear in context. A typical airlaid process begins with an opening step for the fluff pulp and may also include equipment that opens and feeds short cut staple fiber (i.e., synthetic fibers). An airlaid process can also add super absorbent polymers or other powdered solids to the nonwoven web. These materials are suspended in air within a forming system and deposited on a moving forming screen or rotating perforated cylinder. The randomly oriented air-formed batt is bonded by applying latex binder and drying, or by including thermally bonding thermoplastic staple (often “bicomponent” fibers that include an outer layer of low-melting polymer on a higher-melting center) in the web, followed by a thermal bonding step to melt the outer (“sheath”) polymer. Other techniques include hydrogen or embossed bonding, pressure-enhanced hydrogen bonding, hydro-entangling, or combinations of these consolidation techniques.

Airlaid nonwoven fabrics are highly absorbent, lofty fabrics or composites that are relatively cost competitive with similar weight nonwovens because of their use of lower cost wood pulp. Airlaid composites are gaining use for feminine hygiene products (as well as for related products such as wet and dry wipes) and offer a number of potential advantages for baby diapers.

First, using an airlaid core to replace an in situ core is expected to increase machine speeds by a factor of at least about two and a half and potentially by a factor of ten. Stated differently, diaper production at speeds of 800-3000 or more per minute are expected. Second, an airlaid nonwoven structure tends to be more uniform and thus provide a more uniform core. Present in situ, fluff and SAP cores can be less uniform with resulting breakage problems in use. Current cores are not bonded, a factor that can contribute to separation in the end products. As a third advantage, an airlaid core offers the potential for thinner cores at equivalent performance, which arguably increases the baby's comfort and decreases the shelf space needed for retail display. Airlaid cores also could mean fewer machines for handling the fluff, and perhaps most importantly the ability to make a very wide nonwoven structure which can then be cut to the desired size (as opposed to the conventional technique in which the core is made in the size of the individual diaper on a repetitive basis). An airlaid structure can also be readily customized as may be desired or necessary.

The current driving advantage of fluff, however, is its widespread availability and low cost. Accordingly, any improvement in a core structure that is derived from more expensive materials must be balanced against the cost disadvantage. Stated differently, core materials made of materials more expensive than fluff must demonstrate a compensating cost advantage in some other factor such as production speed, machinery cost, or enhanced performance on a weight-for-weight basis.

Accordingly, interest has increased in the potential use of some amount of synthetic fibers such as polyester in cores and potentially in cores formed by airlaid processes. Polyester appears to bring a combination of acquisition and absorption in one layer, an equivalent performance to fluff at lower weight fabrics, improved performance at equivalent weight fabrics, the opportunity to eliminate an acquisition layer in a diaper, and the opportunity to potentially avoid fluff collapse. Synthetic fibers also deliver additional product attributes, such as loft, resilience and the capacity to be embossed.

Although the use of airlaid cores in the feminine hygiene market has recently increased, it is generally accepted that widespread adoption of airlaid diaper cores might represent the most significant potential market. Although some smaller diaper manufacturers are using airlaid cores, the larger manufacturers have not yet participated because the change would mean a significant reconfiguration of their production equipment. A project for a complete airlaid plant could require more than 20,000 engineering hours combined with extensive trials in order to come up with the correct design of the equipment.

Synthetic fibers are presently, however, much more expensive than fluff pulp. Current prices (for comparison purposes) for these materials are about $0.30 per pound for fluff pulp, $0.90 per pound for polyester fiber and SAP, and $1.40 per pound for bicomponent fiber. Synthetic fibers are also packaged and handled differently than fluff (polyester fiber and bicomponent fiber are is typically supplied in bales, fluff is typically supplied as rolls) and thus (as noted above), a change in commercially used cores necessitates a significant capital investment in new equipment. Therefore, an absorbent core that replaces low-cost fluff with higher-cost synthetic materials must offer other cost or performance advantages that offset the higher cost of the starting materials and the potential capital investment.

Accordingly, the need exists for core structures that match or exceed the performance of current fluff and SAP-based cores and that offer compensating advantages in performance, and cost and ease of manufacture.

### SUMMARY OF INVENTION

The present invention is an absorbent core material and does not, standing alone, incorporate cover layers or distribution layers. It can, however, be used with such layers and offers advantages over existing absorbent-core structures, including those that use one or more top sheet distribution layers or other elements. The invention is an airlaid nonwoven structure that includes four components: the fluff; the SAP; a binding structure or element (e.g., bicomponent filaments); and small (2-4 denier) hollow filaments. These four components are blended in the final structure rather than present in separate portions or different layers. This structure permits the amounts of both SAP and bicomponent fiber to be reduced (thus reducing cost) while providing equivalent and often better absorbency.

The foregoing and other objects and advantages of the invention and the manner in which the same are accomplished will become clearer based on the followed detailed description taken in conjunction with the accompanying drawings.

### BRIEF DESCRIPTION OF DRAWINGS

FIGS. 1 through 4 are respective pairs of 20× and 200× micrographs of the core structure according to the present invention.

FIG. 5 is a bar graph showing the relationship between absorbency and the denier of the polyester added according to the present invention when polyester replaces some of the SAP.

FIG. 6 is a bar graph of acquisition versus denier of the added polyester for core structures according to the present invention when polyester replaces some of the SAP.

FIG. 7 is a bar graph of absorbency versus topical treatment of the added polyester according to the present invention when polyester replaces some of the SAP.

FIG. 8 is a plot comparing absorbency versus fabric weight for conventional core structures and core structures according to the present invention when polyester replaces some of the fluff.

### DETAILED DESCRIPTION

The invention is an absorbent core structure for acquiring and absorbing aqueous-based liquids in hygiene products. The core structure comprises a macroscopically homogeneous nonwoven blend of fluff pulp for wicking and absorbing aqueous-based liquids, a super absorbent polymer for absorbing aqueous-based liquids and retaining the liquid against moderate pressure exerted against the core structure, a binding structure or element for providing at least some of the bonding in the nonwoven blend, and a plurality of small denier hollow filaments for enhancing the moisture transport and storage of the liquid and improving the physical structure of the core as compared to the equivalent amount of fluff.

As used herein, the phrase “binding structure or element” refers to the use of a method (e.g., hydrogen bonding), or an element (e.g., synthetic bicomponent fibers), or a combination of these, to increase the structural integrity of the absorbent core structure. As noted in the Background such binding can also include (but is not limited to) pressure-enhanced hydrogen bonding, latex binders, and hydro-entangling techniques.

The fluff pulp is preferably present in an amount of between about 30 and 80 percent by weight of the core structure with amounts of between about 40 and 55 percent by weight more preferred. Fluff pulp is available from a variety of sources, with Weyerhaeuser and Georgia-Pacific being two of the largest U.S. producers. Fluff pulp's properties and the manner of including it (and the other components) in an airlaid process are well understood and need not be elaborated on herein. Appropriate airlaid machinery is available from well known and well established companies such as M&J Fibretech A/S of Horsens, Denmark and Dan-Web (Dan-Webforming International A/S, Risskov, Denmark).

The core structure is preferably formed at a basis weight of between about 200 and 800 grams per square meter (gsm). In one sense, there are no strict upper and lower limits for basis weight. In general, and all other factors being equal, total absorption is directly proportional to fabric weight. Thus, reducing the basis weight reduces the total capacity. Alternatively, for a given density, thickness is likewise directly proportional to basis weight, so that fabrics become thicker as their basis weight increases. Therefore, core structures for absorbent items are typically selected to provide a desired combination of absorption and thickness.

In preferred embodiments, basis weights of between about 500 and 600 gsm are most preferred.

The super absorbent polymer is preferably present in an amount of between about five and 60 percent by weight, and more preferably between about 25 and 50 percent by weight. The nature, structure, chemistry, and availability of super absorbent polymers are likewise, well understood in the art, and can be selected by those of ordinary skill in this art without undue experimentation. Super absorbent polymers for this and a variety of related purposes are available from companies such as Stockhausen, Inc., Greensboro, NC (FAVOR^{Â}® SAPs) and The Dow Chemical Company (DRYTECH^{Â}® SAPs).

Superabsorbent polymers (“SAPs”) are generally defined as dry materials which can spontaneously absorb an aqueous fluid at a rate of at least twenty times their own weight, e.g. U.S. Pat. No. 6,277,772, citing Absorbent Polymer Technology, Studies in Polymer Sciences 8, Elsevier 1990.

Acrylic acid is the most typical base monomer for SAPs. Any water soluble electrolyte polymer is conceptually suitable for producing an SAP (e.g., amine-based monomers), but acrylic acid is preferred commercially for reasons of safety, cost and performance. As well recognized by those familiar with these polymers, the double bond in acrylic acid can unfold to form the polymer backbone, with the acid (carboxyl) groups forming pendant functional groups.

For a number of reasons, a suitable SAP contains a desired amount of cross-linking and a desired amount of fully charged functional groups; i.e., groups carrying a full charge rather than just a polar structure. Thus, SAPs are a subgroup of a more general classification referred to as “polyelectrolytes” see Lewis, Hawley's Condensed Chemical Dictionary, 12^{th} Ed., Van Nostrand Reinhold 1993 at page 932. Polyelectrolytes that are not cross-linked are simply soluble in water. When sufficiently cross-linked, however, the polyelectrolytes become insoluble. Nevertheless, their polar character, their charged functional groups, and the polar character of water, maintain the mutual attraction between the polyelectrolytes and water. This mutual attraction enables the cross-linked polyelectrolytes to absorb water instead of dissolving in it. The hypothetical goal is to cross-link the molecules so extensively that a single particle of the SAP is essentially one molecule.

In order to create charged functional groups rather than merely polar ones, the polyacrylic acid is partially neutralized, typically with a base such as sodium hydroxide to form water and the appropriate salt of the acid functional group. The resulting full charge on the functional group significantly increases the absorption characteristics of the material.

The gel volume of an SAP depends upon the amount of charge and the degree of cross Once the degree of cross-linking is fixed, the weight-to-weight absorption of the material will increase based on the degree of neutralization. As an example, an SAP that is approximately 70 percent neutralized can absorb approximately 30 times its weight in water. If not neutralized, however, the same composition with the same degree of cross-linking will absorb less than five times its weight in water. In short summary, the anions or cations attract the water to the SAP while the cross-linking holds it in place.

The synthetic bicomponent filament is present in an amount sufficient to provide bonding in the nonwoven blend, but less than an amount that would overly stiffen the resulting nonwoven blend or undesirably reduce its tensile strength. In the case of the bicomponent filament and the small denier hollow filaments, the increased amount of synthetic fiber can reduce the tensile strength of the core by reducing the hydrogen bonding that exists between the pulp fibers. Accordingly, the bicomponent filament is preferably present in an amount of between about five and 20 percent by weight of the core structure, and more preferably in an amount of between about 10 and 15 percent by weight. The bicomponent fibers will typically comprise a polyester or polypropylene core and a polymer sheath formed from a polymer having a lower melting temperature than the core. In preferred embodiments, the sheath portion of the bicomponent fiber is selected from the group consisting of polyethylene, polyethylene copolymers, polypropylene, polyester copolymers, and blends of polyester and polyethylene. Bicomponent suitable for use in the invention is available from a number of sources including KoSa (“Celbond”), Wellman International (“Wellbond”), TREVIRA and ES Fibervisions. The nature and performance of bicomponent fibers are likewise well understood in this art, and an appropriate choice can be made by those of ordinary skill for desired purposes and without undue experimentation.

The most interesting feature of the invention is the use of the plurality of small denier hollow filaments for enhancing the moisture transport and storage of the liquid and for improving the physical structure as compared to the fluff. In preferred embodiments, the hollow filaments are formed of polyester. They are preferably present in an amount of between about 5 and 20 percent by weight of the core structure, and more preferably between 5 and 15 percent by weight. Round cross sections are convenient in terms of manufacturing the hollow filaments, but the hollow filaments are not required to be round, and non-round cross sections can also be incorporated.

It has been further discovered, according to the present invention, that contrary to conventional expectations, the smaller denier hollow filaments present unexpected advantages in absorptive capacity and moisture retention as will be discussed with respect to the drawings herein. Thus, the hollow filaments preferably have a denier of less than 15, more preferably less than 6, most preferably denier of between about 2 and 4, and in some cases a denier as low as 1 dpf.

The acquisition and retention performance of smaller denier (ie., 6 dpf or less) fibers is generally unexpected. In general, low denier fibers give more closed, more dense webs that are expected to take up liquid slowly than more open webs formed from higher denier (e.g., 6-15 dpf or higher) fibers. Similarly, for a given dpf, hollow fibers are conventionally expected to wick more slowly than solid ones.

The hollow filaments preferably have a staple length sufficient to exhibit fiber properties, but less than the length that would fail to complement the fluff in the structure. Stated differently, a structure that has a length too similar to its width will not exhibit the physical properties that are desirable for fibers. Although the term fiber does not have an exact limiting definition, it is generally well understood that a fiber should a length-to-width ratio of at least about 100; e.g., Tortora, Fairchild's Dictionary of Textiles, 7th Edition (1996) at page 214.

From a fiber behavior standpoint, there is generally no negative upper limit to fiber length for the hollow filaments. In that sense, the goal is to incorporate the longest fiber that actual airlaid forming and fabric production will allow, as greater length always improves fabric performance, from a strength, bulk and moisture management standpoint. The limitations are always on the high side, and are related to actual forming of the airlaid web at the forming head and getting the fiber through the screens, where longer fibers are harder to move through at maximized throughput. Older machines run best with 3 mm as the maximum cut length, somewhat newer machines can run 4 mm, and the newest machines can comfortably run 6 mm cut length. Some vendors quote longer cut lengths as being possible, but there is limited commercial evidence of these to date.

In the case of the present invention, however, the hollow polyester fiber is present with, and must compliment the fluff pulp in the nonwoven composite structure. Thus, the synthetic fibers present, both bi-component and hollow, are preferably about the same length as the average length of the wood pulp, and most preferably about the same length as the longer (i.e., above-average) fluff lengths. Matching the fiber lengths also provides an advantage in using the machinery used to make the nonwoven structure in which the sizes are best kept similar to one another. Accordingly, the hollow filament preferably comprises cut staple fibers having a length of between about three and six millimeters; fluff pulp, having a typical length of about 3 mm.

Although the preferred embodiments are discussed herein in terms of hollow polyester filaments, it will be understood that the small denier and hollow construction are functionally advantageous, and that small denier hollow filaments of polymers, other than polyester, are expected to provide similar advantages.

The hollow polyester fibers also preferably have a hydrophilic surface, with a permanent hydrophilic treatment being the preferred method of obtaining the hydrophilic surface.

In other preferred embodiments, the hollow polyester filament can be fractured or produced by the method set forth in co-pending and commonly assigned application Serial No. 10/065,436 filed Oct. 17, 2002 for “Highly Absorbent Polyester Fibers,” the contents of which are incorporated entirely herein by reference. Similarly, the use of various amounts of polyethylene glycol and pentaeyrthritol has provided significant advantages in polyester characteristics and performance, and appropriate discussions of these are set forth in commonly assigned U.S. Pat. Nos. 6,509,091; 6,485,829; 6,454,982; 6,399,705; 6,322,886; 6,303,739; 6,294,254; 6,291,066; and 6,214,270, and published applications Nos. 20020019508; 20010039160; 20010029281; 20010027244; 20010003131; and 20010002737, all of which are incorporated entirely herein by reference.

The resulting product is an airlaid core structure with a basis weight of between about 200 and 800 grams per square meter. As will be discussed with respect to the drawings, however, the invention a method of obtaining superior performance at equivalent basis weights for airlaid nonwoven composite structures, or of obtaining similar performance at lower basis weights. Thus, where equivalent performance is desired or required, the invention provides a way of doing such at lower costs. Alternatively, if basis weight is the limiting factor, the invention will provide greater performance.

The core structure described and claimed herein is fundamental to a number of structures, more than can be easily enumerated, and the uses of which are as varied as the need to absorb liquids in a number of circumstances. Accordingly, the uses described herein are exemplary, rather than limiting, of the uses of the core structures of the present invention and those of skill in this art will be able to develop equivalents without undue experimentation that will fall within the scope of the invention and the claims.

With that in mind, most typical absorbent structures include at least a top sheet, a back sheet, and one or more performance layers therebetween. Accordingly, in this aspect the invention comprises such an absorbent structure with a porous top sheet, a back sheet, and a core between the back sheet and the top sheet that comprises the fluff pulp, the super absorbent polymer, the bicomponent filament and the small denier hollow polyester fibers.

As known to those familiar with these materials, and as set forth in numerous issued patents, the porous top sheet (regardless of its particular name in particular disclosures) serves as a gathering mechanism for fluid and for passing it to the core where it can be absorbed and retained. The back sheet, in contrast, is usually preferably selected to be a fluid barrier so as to prevent liquid from leaking through it. A baby diaper, is of course, most exemplary of this in that the top sheet is positioned next to the child to receive fluids, while the back sheet forms the exterior of the diaper which is desirably kept as dry as possible.

Many absorbent structures also include one or more distribution layers between the top sheet and the core, as well as more than one core layer. As the name implies, a distribution layer serves to primarily or exclusively spread liquid that strikes the top sheet in a small area over a larger area of the core, thus enhancing the core's ability to absorb and retain a larger volume of liquid.

It will be understood that the invention can be used in conjunction with any one or more of these additional elements.

In addition to baby diapers, other types of structures within the scope of the present invention include, but are not limited to, adult incontinence garments, training pants, swim wear, absorbent underwear, baby wipes, medical absorbent pads, and feminine hygiene pads.

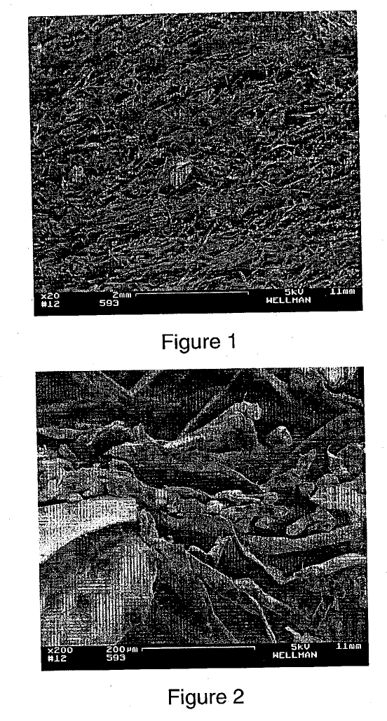

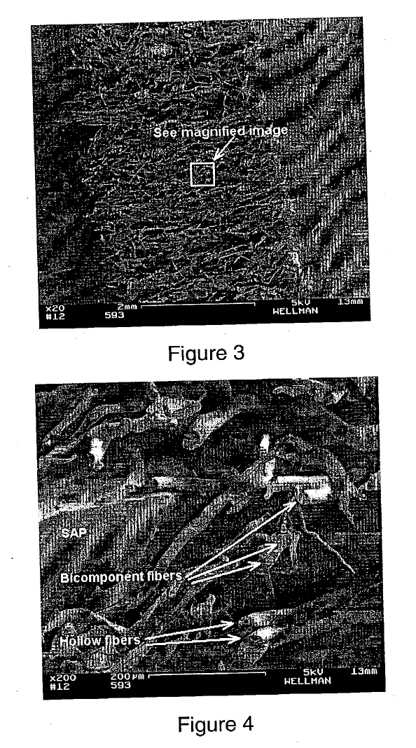

FIGS. 1 through 4 are paired sets of micrographs of structures according to the invention taken at respective magnifications of 20× and 200×. Thus, FIG. 2 is an enlarged portion of FIG. 1, and FIG. 4 is an enlarged portion of FIG. 3. The 20× micrographs include a millimeter bar for comparison purposes, and the 200× photographs include a 200 micron bar for comparison purposes.

The 200× photographs all illustrate in greater detail the four components of the preferred embodiment of the invention and these are particularly labeled in the pairing of FIGS. 3 and 4. Thus, the photographs show the super-absorbent polymer, the bi-component fibers, the hollow fibers, and the fluff pulp.

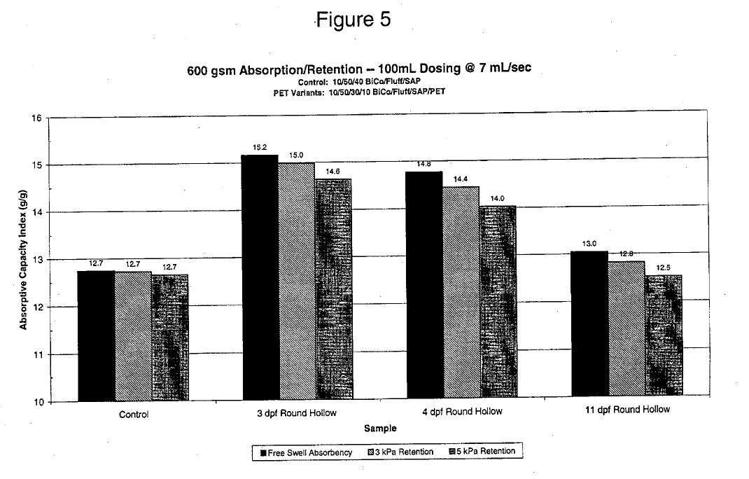

FIGS. 5-8 plot various characteristics of structures according to the present invention in relation to control structures. For comparison purposes, all of the structures represented by FIGS. 5-8 have the same density of 0.120 grams per cubic centimeter (g/cm³).

FIG. 5 is a plot of the absorptive capacity on a gram per gram basis for four different cores: a control, and three according to the present invention. Tests for absorptive capacity are generally well known in the art. U.S. Pat. No. 5,124,188 (Roe et al), issued Jun. 23, 1992 at Columns 27-28 (herein incorporated by reference) gives a description of one version of an AbsorptiveCapacity test. Tests set forth by Kimberly-Clark or Proctor & Gamble (e.g. U.S. Pat. Nos. 6,429,350 or 6,217,889), can be satisfactorily used to illustrate the same trends in the data. Another suitable test is the “Standard Test Method for Absorbency Time, Absorbency Capacity, and Wicking Time,” published by INDA, Association of the Nonwoven Fabrics Industry, Cary, N.C.

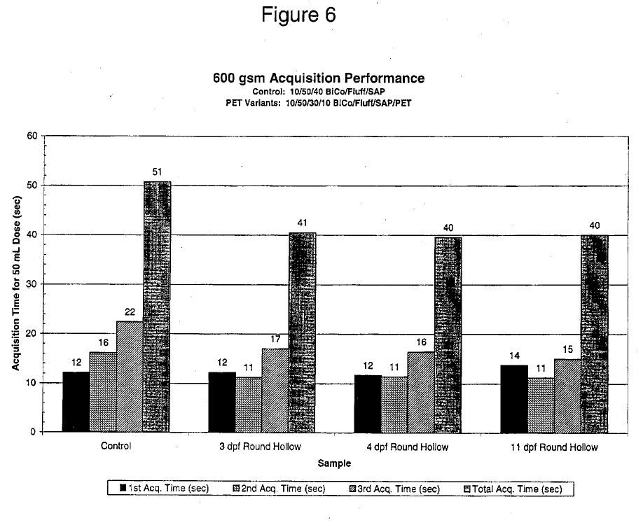

Stated differently, the trend exhibited in FIG. 5 (and the trends exhibited in FIGS. 6-8) will be evident based on a number of different tests for absorptive capacity and those of skill in the art will be able to demonstrate the same results (in comparative terms) using any appropriate test.

In particular, FIG. 5 shows the denier trend using hollow polyester fibers to replace super absorbent polymer in an amount of ten percent in core structures of identical density (0.120 g/cm³). Thus, the control core structure of FIG. 5 is formed of a 10/50/40 percent by weight combination of bi-component filament/fluff/SAP. The polyester-containing examples are formed of a combination of 10/50/30/10 percent by weight of bi-component fiber/fluff/SAP/hollow polyester filament. All of the structures had the same basis weight of 600 gsm. The test measures how much liquid the fabric will hold upon no load (also referred to as free swell), under 3 KPa pressure, and under 5 KPa pressure (1 psi=6.895 Kpa=0.06895 bar). As noted above, the tests are based on prior established techniques and have a significant level of industry acceptance. In general 100 milliliters of 0.9 percent saline solution is delivered to the fabric at a rate of 7 milliliters per second.

The pad is then measured to determine how much it holds. A 3 KPa weight is then added and the amount that stays in is recorded, following which a 5 KPa weight is added with the amount retained again observed. The Y-axis shows the amount of fluid held divided by the actual weight of the pad tested. Thus, a higher absorptive capacity (taller portions of the bar graphs) represent the best results in this test.

The data plotted in FIG. 5 shows that the smallest denier of hollow filament (3 and 4 denier) gave the best absorption/retention results. larger hollow filament (11 dpf in FIG. 5) showed a slight benefit over the control in free swell, but a statistically meaningless difference in 3 kPa and 5 kPa retention.

FIG. 6 is a bar graph of acquisition performance versus denier. Each fabric was a 600 gram per square meter (gsm) airlaid nonwoven with the control fabric again being a 10/50/40 (percent) composite of bi-component fiber/fluff/SAP. The polyester-containing versions were 10/50/30/10 (percent) blends of bi-component fiber/fluff/SAP/(hollow) polyester fibers.

In this test, a 50 milliliter sample (also referred to as an “insult”) of synthetic urine is released into the fabric from a point source and the time for the fabric to acquire the liquid is recorded. After a 30 minute wait, another sample is added and the time for acquisition is again recorded. Finally, after 30 more minutes a third sample is delivered. The last column for each sample is the sum of the acquisition times. In particular, FIG. 6 illustrates that all of the tested deniers of hollow fibers improved the acquisition time when replacing SAP in an amount of 10 percent, and that all of the deniers performed substantially the same as one another.

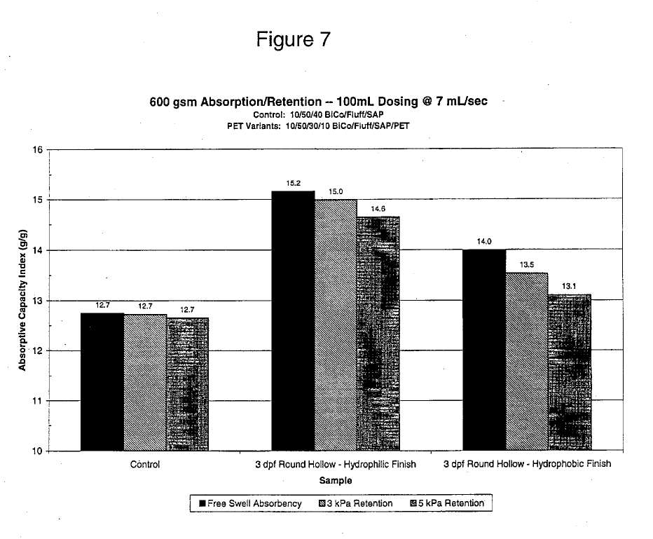

FIG. 7 illustrates the results of another test of absorptive capacity for the same fabrics (control and invention) as illustrated in FIG. 5 but with a hydrophilic finish being applied to the polyester fibers in one of the samples according to the invention and a hydrophobic finish being applied to the other. Once again, the absorbent capacity was tested and reported as against the control and these two other fabrics. FIG. 7 illustrates that a hydrophobic finish on a 3 dpf fiber produces better results than the control structure, and that a hydrophilic finish exhibits the best results of the three samples.

The use of finishes on synthetic filaments is well understood in the art, and can be practiced by those of skill in this art without undue experimentation. Exemplary (but not limiting) hydrophilic finishes for polyester (and other) filaments include esters of glycerol and fatty acids, silicone and related compounds, and certain acrylate and other monomers.

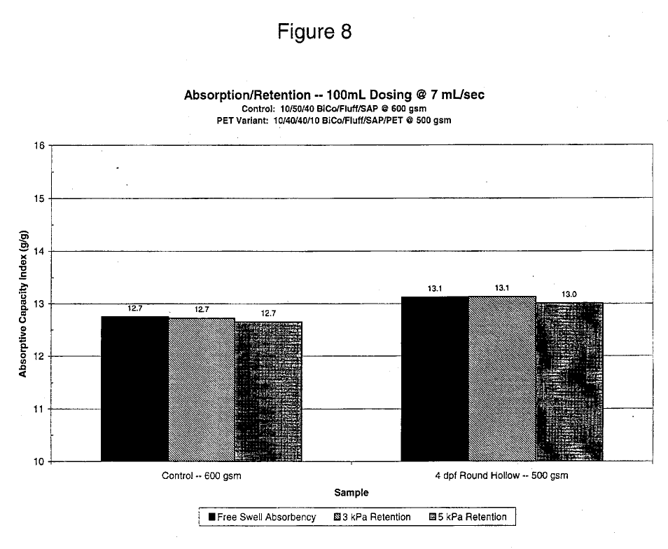

FIG. 8 also presents absorptive capacity results, and specifically illustrates the effect of reducing the basis weight of the fabric. In the tests and results illustrated by FIG. 8, the control fabric was a 10/50/40 percent by weight blend of bicomponent/fluff/SAP, and the fabric according to the invention was a 10/40/40/10 percent by weight bicomponent/fluff/SAP/hollow polyester of 4 dpf and (as in all cases herein, a round cross section).

The fabric according to the invention, however, was formed into a 500 gsm basis weight as compared to the 600 gsm basis weight of the control. Accordingly, FIG. 8 illustrates that the basis weight can be significantly reduced while still providing the equivalent absorbency on a gram for gram basis. With respect to the specific samples illustrated in FIG. 8, the cost savings would be about ten percent to obtain the same results as the control. Alternatively, fabrics according to the invention having the same basis weight as the control (e.g. FIG. 5) exhibit a significant improvement in absorptive capacity.

In the drawings and specification there has been set forth a preferred embodiment of the invention, and although specific terms have been employed, they are used in a generic and descriptive sense only and not for purposes of limitation, the scope of the invention being defined in the claims.

1. An absorbent core structure for acquiring and absorbing aqueous-based liquids in hygiene products, said core structure comprising:
a nonwoven blend of,
fluff pulp for wicking and absorbing an aqueous-based liquid;
a superabsorbent polymer for absorbing an aqueous-based liquid and retaining the liquid against moderate pressure against said core structure;
a synthetic bicomponent filament for providing at least some of the bonding in said nonwoven blend; and
a plurality of small denier hollow filaments for enhancing the moisture transport and storage of the liquid and improving the physical structure as compared to fluff.

2. A core structure according to claim 1 wherein said hollow filaments comprise polyester.

3. A core structure according to claim 1 wherein said bicomponent filament is present in an amount sufficient to provide bonding in said nonwoven blend, while less than the amount that overly-stiffens the resulting nonwoven blend.

4. A core structure according to claim 1 wherein said bicomponent filament is present in an amount of between about 5 and 20 percent by weight of said core structure.

5. A core structure according to claim 1 wherein said small denier hollow filament is present in an amount of between about 5 and 20 percent by weight of said core structure.

6. A core structure according to claim 1 wherein said hollow filaments have a denier less than 6.

7. A core structure according to claim 1 wherein said hollow filaments are between about 2 and 4 denier.

8. A core structure according to claim 1 wherein said super absorbent polymer is present in an amount of between about 5 and 60 percent by weight of the core structure.

9. A core structure according to claim 1 wherein said fluff is present in an amount of between about 30 and 80 percent by weight of the core structure.

10. A core structure according to claim 1 wherein said hollow filaments are present in an amount sufficient to improve the loft of the structure as compared to fluff alone.

11. A core structure according to claim 1 wherein said hollow filament is polyester and present in an amount of between about 5 and 20 percent by weight of the core structure.

12. A core structure according to claim 1 wherein said hollow filament has a staple length sufficient to exhibit fiber properties, while less than the length that would fail to complement the fluff in said structure.

13. A core structure according to claim 12 wherein said hollow filament has substantially the same length as the fibers of said fluff.

14. A core structure according to claim 1 wherein said hollow filament is polyester and comprises cut staple fibers having a length between about 3 and 6 millimeters.

15. A core structure according to claim 1 wherein said bicomponent filaments comprise a core selected from the group consisting of polyester and polypropylene and a sheath having a lower melting temperature than said core.

16. A core structure according to claim 15 wherein said sheath is selected from the group consisting of polyester, polyethylene, polyethylene copolymers, polypropylene, polyester copolymers, and blends thereof.

17. An airlaid core structure according to claim 1.

18. A core structure according to claim 1 wherein said hollow filaments comprise polyester staple fibers with a hydrophilic surface.

19. A core structure according to claim 1 wherein said hydrophilic surface comprises a hydrophilic finish.

20. A core structure according to claim 1 wherein said hollow filament comprises a fractured polyester filament.

21. A core structure according to claim 1 having a basis weight of between about 200 and 800 grams per square meter.

22. A core structure according to claim 1 having a basis weight of between about 500 and 600 grams per square meter.

23. A core structure according to claim 1 wherein said hollow filaments have a round cross-section.

24. A core structure according to claim 1 wherein said hollow filaments have a non-round cross-section.

25. An absorbent core structure for acquiring and absorbing aqueous-based liquids in hygiene products, said core structure comprising:
a nonwoven composite blend of
between about 5 and 60 percent by weight of a superabsorbent polymer;
between about 5 and 20 percent by weight of a synthetic bicomponent filament for providing at least some of the bonding in said nonwoven blend;
between about 5 and 20 percent by weight of 2-4 denier hollow polyester filaments for enhancing the acquisition and absorbance of the liquid and improving the physical structure as compared to fluff; and
the remainder as fluff pulp.

26. An absorbent core structure according to claim 25 comprising:
between about 40 and 55 percent by weight fluff pulp;
between about 25 and 50 percent by weight of a superabsorbent polymer;
between about 10 and 15 percent by weight of a synthetic bicomponent filament for providing at least some of the bonding in said nonwoven blend; and
between about 5 and 15 percent by weight of said polyester filaments.

27. A core structure according to claim 25 wherein said hollow filament is polyester and comprises cut staple fibers having a length between about 3 and 6 millimeters.

28. A core structure according to claim 25 wherein said bicomponent filaments comprise a core selected from the group consisting of polyester and polypropylene and a sheath selected from the group consisting of polyester, polyethylene, polyethylene copolymer, polypropylene, polyester copolymers, and blends thereof.

29. An airlaid core structure according to claim 25.

30. A core structure according to claim 25 wherein said hollow filaments include a hydrophilic finish.

31. A core structure according to claim 25 wherein said hollow filament comprises a fractured polyester filament.

32. A core structure according to claim 25 having a basis weight of between about 200 and 800 grams per square meter.

33. A core structure according to claim 25 having a basis weight of between about 400 and 600 grams per square meter.

34. A core structure according to claim 25 wherein said hollow filaments have a round cross-section.

35. An absorbent structure comprising:
a porous top sheet;
a substantially liquid impenetrable back sheet; and
an absorbent core between said back sheet and said top sheet, said core comprising a nonwoven composite blend of between about 5 and 60 percent by weight of a superabsorbent polymer;
between about 5 and 20 percent by weight of a synthetic bicomponent filament for providing at least some of the bonding in said nonwoven blend;
between about 5 and 20 percent by weight of 2-4 denier hollow polyester filaments for enhancing the acquisition and absorbance of the liquid and improving the physical structure as compared to fluff; and
the remainder as fluff pulp.

36. An absorbent structure according to claim 35 comprising a baby diaper.

37. An absorbent structure according to claim 35 comprising an adult incontinence garment.

38. An absorbent structure according to claim 35 comprising an incontinence pad.

39. An absorbent structure according to claim 35 comprising a feminine hygiene pad.

40. An absorbent structure according to claim 35 comprising training pants.

41. An absorbent structure according to claim 35 comprising swim wear.

42. An absorbent structure according to claim 35 comprising absorbent underwear.

43. An absorbent structure according to claim 35 comprising a medical absorbent.

44. An absorbent core structure according to claim 35 wherein said absorbent core comprises:
between about 40 and 55 percent by weight fluff pulp;
between about 25 and 50 percent by weight of a superabsorbent polymer;
between about 10 and 15 percent by weight of a synthetic bicomponent filament for providing at least some of the bonding in said nonwoven blend; and
between about 5 and 15 percent by weight of said polyester filaments.

45. An absorbent structure according to claim 35 wherein said hollow polyester filament comprises cut staple fibers having a length between about 3 and 6 millimeters.

46. An absorbent structure according to claim 35 wherein said bicomponent filaments comprise a core selected from the group consisting of polyester and polypropylene and a sheath selected from the group consisting of polyethylene, polyethylene copolymer, polypropylene, polyester copolymers, and blends thereof.

47. An airlaid absorbent structure according to claim 35.

48. An absorbent structure according to claim 35 wherein said hollow filaments include a hydrophilic finish.

49. An absorbent structure according to claim 35 wherein said hollow polyester filament comprises a fractured filament.

50. An absorbent structure according to claim 35 having a basis weight of between about 200 and 800 grams per square meter.

51. An absorbent structure according to claim 35 having a basis weight of between about 400 and 600 grams per square meter.

52. An absorbent structure according to claim 35 wherein said hollow filaments have a round cross-section.

53. An absorbent core structure for acquiring and absorbing aqueous-based liquids in hygiene products, said core structure comprising:
a nonwoven composite blend of
between about 5 and 60 percent by weight of a superabsorbent polymer;
between about 5 and 20 percent by weight of 2-4 denier hollow polyester filaments for enhancing the acquisition and absorbance of the liquid and improving the physical structure as compared to fluff;
a binding element; and
the remainder as fluff pulp.

54. A core structure according to claim 53 wherein said binding element comprises between about 5 and 20 percent by weight of a synthetic bicomponent filament.

55. A core structure according to claim 53 wherein said binding element comprises hydroentangled fibers.

56. A core structure according to claim 53 wherein said binding element comprises hydrogen bonding.

57. A core structure according to claim 53 wherein said binding element comprises pressure-enhanced hydrogen bonding.

58. A core structure according to claim 53 wherein said binding structure comprises latex.

59. A core structure according to claim 53 wherein said hollow filament is polyester and comprises cut staple fibers having a length between about 3 and 6 millimeters.

60. A core structure according to claim 53 wherein said bicomponent filaments comprise a core selected from the group consisting of polyester and polypropylene and a sheath selected from the group consisting of polyester, polyethylene, polyethylene copolymers, polypropylene, polyester copolymers, and blends thereof.

61. An airlaid core structure according to claim 53.

62. A core structure according to claim 53 wherein said hollow filaments include a hydrophilic finish.

63. A core structure according to claim 53 wherein said hollow filament comprises a fractured polyester filament.

64. A core structure according to claim 53 having a basis weight of between about 200 and 800 grams per square meter.

65. A core structure according to claim 53 having a basis weight of between about 400 and 600 grams per square meter.

66. A core structure according to claim 53 wherein said hollow filaments have a round cross-section.
